(19) United States
(12) Patent Application Publication
(10) Pub. No.: US 2018/0110552 A1
(43) Pub. Date: Apr. 26, 2018

(54) Interconnecting Cannulated Soft Tissue Screw System and Related Methods

(71) Applicant: SangDo Park, Los Angeles, CA (US)

(72) Inventor: SangDo Park, Los Angeles, CA (US)

(21) Appl. No.: 15/622,359

(22) Filed: Jun. 14, 2017

Related U.S. Application Data
(60) Provisional application No. 62/410,850, filed on Oct. 21, 2016.

(51) Int. Cl. A61B 17/86

(52) U.S. Cl. CPC A61B 17/8685; A61B 17/864; A61B 17/8625; A61B 17/8695

(57) ABSTRACT

Disclosed is an apparatus and related methods for soft tissue fixation to bone by use of interconnecting cannulated screws. An anchor screw directly purchases into the bone, and a washer screw threads into the anchor screw via a cannulated mechanism, fixing the interposed soft tissue onto the bone.

### CROSS-REFERENCE TO RELATED APPLICATIONS

This document claims the benefit and priority of U.S. Prov. Pat. App. Ser. No. 62/410,850 (filed Oct. 21, 2016) by Dr. SangDo Park, M.D. for “Interconnecting Cannulated Soft Tissue Screw System.”

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC (CD) & INCORPORATED BY REFERENCE OF THE MATERIAL ON THE CD

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the field of orthopedic surgery and, in particular, the interconnection of soft tissue to bone.

### Background of the Invention

Anatomically, soft tissue can refer to tissues that connect, support, or surround human-bodily structures and organs. Soft tissue contrasts with hard tissue of the human-body, such as bones. Soft tissues are often connected to bone for ideal operation of the human body and, when soft tissues inadvertently disconnect from bone, orthopedic surgery can be required for reconnection.

Currently, the most widely used method of soft tissue fixation to bone is via a suture anchor or other similar devices. Suture anchors are made up of: (a) an anchor portion is set into a bore drilled into the bone (e.g., via a screw mechanism or interference fit mechanism); (b) a suture portion that is configured to grip soft tissue; and (c) an eyelet or connection for linking the anchor portion to the suture portion. The suture anchor method requires placing grasping sutures onto the soft tissue and has limited fixation strength in hollow bone (such as that in diaphyseal bone encountered in subpectoral biceps tenodesis surgery). Typical suture anchors can also be tedious and time-consuming to install.

### SUMMARY OF THE INVENTION

In view of the foregoing, an object of this specification is to disclose an apparatus and related methods of connecting soft tissue to bones with improved fixation strength in hollow bones. Disclosed may be an apparatus and related methods of attaching soft tissue to bone via use of interconnecting cannulated screws. Suitably, the disclosed interconnecting cannulated screws may be applied to any circumstance which requires attachment of soft tissue to bone. In the preferred embodiment, interconnecting cannulated screws comprise: (a) an anchor screw with male threads and a threaded female receptacle; (b) a washer screw with male threads and a head; and (c) a washer that couples to the washer screw. Suitably, the female receptacle of the anchor screw and the male threads of the washer screw define a cannulated mechanism. In one mode of use, the anchor screw directly purchases into the bone while the washer screw is inserted through the washer and penetrated through the soft tissue before ultimately being threaded into the female receptacle of the anchor screw. In a preferred embodiment, the washer screw can be guided by a guide needle through the washer, soft tissue, and anchor screw. When complete, the soft tissue is interposed between the washer and the anchor screw while the washer screw holds tast the interposition of the soft tissue. Suitably, the disclosed cannulated mechanism fixes the interposed soft tissue onto the bone.

The interconnecting cannulated screws offer the advantages over suture anchors of speed, ease of use, use in both diaphyseal and metaphyseal bones, and bypasses the need for placing grasping sutures into the soft tissue (which is a step necessary in most current fixation methods). The preferred method allows for capturing of the soft tissue via direct soft tissue penetration and can be used for both small and large tendons and ligaments. Furthermore, the anchor screw can be placed as a cancellous screw or a cortical screw depending on the region of the bone where fixation is desired. In one embodiment, the interconnecting cannulated screws also allow for easier interposition of materials impregnated with biologics into the repair.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached Figures in which:

FIG. 1A is a perspective view of a washer;

FIG. 1B is a side view of the washer;

FIG. 1C is a cross-section of the washer;

FIG. 2A is a perspective view of a washer screw;

FIG. 2B is a side view of the washer screw;

FIG. 2C is a cross-section of the washer screw;

FIG. 3A is a perspective view of an anchor screw;

FIG. 3B is a side view of the anchor screw;

FIG. 3C is a cross-section of the anchor screw;

FIG. 4 is a view of a guide needle;

FIG. 5 is a schematic of the guide needle traversing the anchor screw and the washer screw, while the washer screw traverse the soft tissue and the washer, and while the anchor screw is deposited into a bone;

FIG. 6 is a further schematic of the guide needle traversing the anchor screw and the washer screw, while the washer screw traverse the soft tissue and the washer, and while the anchor screw is deposited into a bone; and,

FIG. 7 is yet a further schematic of the guide needle traversing the anchor screw and the washer screw, while the washer screw traverse the soft tissue and the washer, and while the anchor screw is deposited into a bone.

It is to be noted, however, that the appended Figures illustrate only typical embodiments of this invention and are therefore not to be considered limiting of its scope, for the invention may admit to other equally effective embodiments that will be appreciated by those reasonably skilled in the relevant arts. Also, Figures are not necessarily made to scale but are representative.

### DETAILED DESCRIPTION OF PREFERRED EMBODIMENTS

Disclosed may be an apparatus and related methods of connecting soft tissue to bones with improved fixation strength in hollow bones. Specifically, disclosed are methods of attaching soft tissue to bone via use of interconnecting cannulated screws. In the preferred embodiment, interconnecting cannulated screws comprise: (a) an anchor screw with male threads and a threaded female receptacle; (b) a washer screw with male threads and a head; and (c) a washer that couples to the washer screw. Suitably, the female receptacle of the anchor screw and the male threads of the washer screw define a cannulated mechanism. Suitably, the disclosed interconnecting cannulated screws may be applied to any circumstance which requires attachment of soft tissue to bone. The more specific details of the interconnecting cannulated screws are disclosed in connection with the Figures.

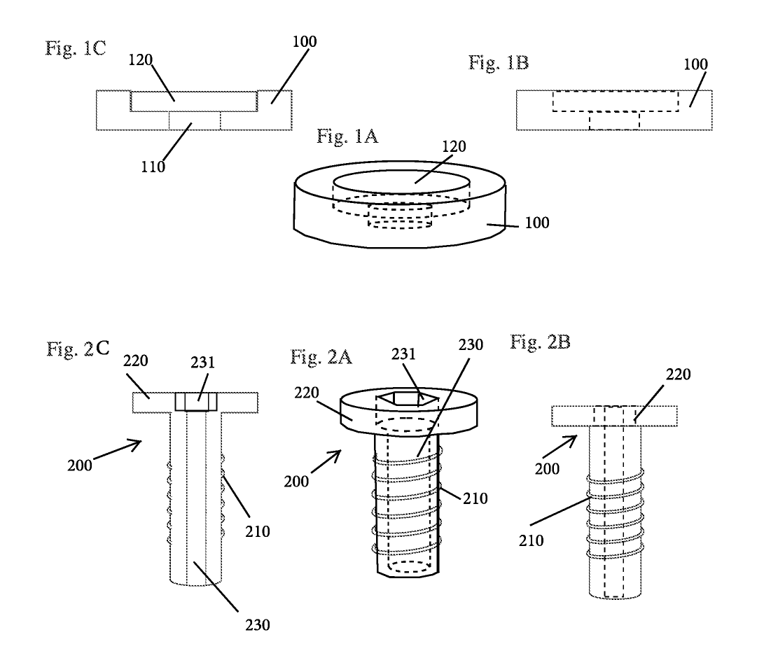

FIG. 1A is a perspective view of a washer 100 with internal features drawn in broken lines. FIG. 1B is a side view of the washer 100 with internal features shown in broken lines. FIG. 1C is a cross-section of the washer 100 to illustrate the internal features of the washer 100. As shown, the washer 100 preferably defines a disc and features a central aperture 110 and a recessed area 120. In use, as discussed below, the central aperture 110 may femininely receive the male threads (210, FIG. 2A) of the washer screw (200, FIG. 2A) while the recessed area 120 of the washer 100 may receive and abut the head (220, FIG. 2A) of washer screw (200, FIG. 2A).

FIG. 2A is a perspective view of a washer screw 200 with a head 220, male threads 210, and a canal or bore 230 (shown in broken lines) through its length. FIG. 2B is a side view of the washer screw 200. FIG. 2C is a cross-section of the washer screw 200 to illustrate the canal 230. As shown, washer screw 200 which is cannulated along its entire length by the canal or bore 230. The screw 200 is comprised of head portion 220, bore portion 230, and male threads 210. As shown, the bore portion 230 features a hexagonal portion 231 so that the washer screw 200 may be driven, as discussed below, into the anchor screw 300 (see FIG. 7) via a screw driver or wrench (e.g., Allen wrench) with a corresponding male hexagonal structure.

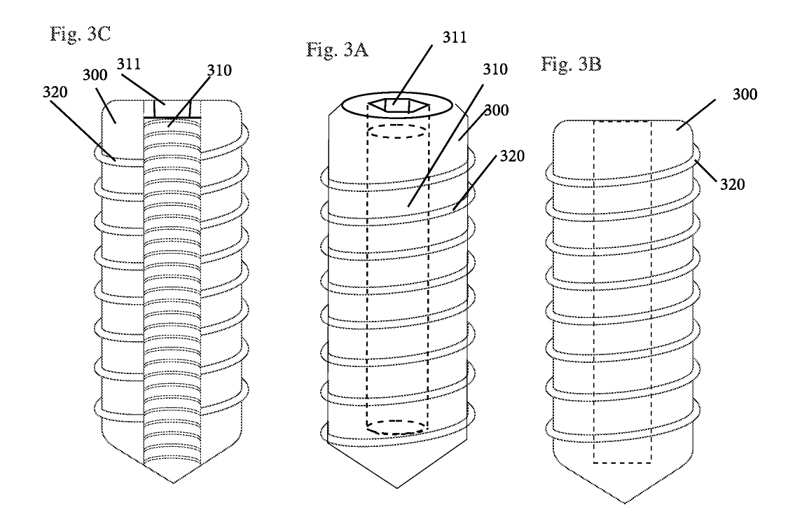

FIG. 3A is a perspective view of an anchor screw 300 with male threads 320 and an internal canal or bore 310 (shown in broken lines). FIG. 3B is a side view of the anchor screw 300. FIG. 3C is a cross-section of the anchor screw 300 to illustrate the bore or canal 310. The anchor screw 300 is comprised of a threaded bore portion (310, FIG. 3C) and external threads (320, FIG. 3A). The external threads 320 can be designed as either cancellous or cortical depending on the specific region of the bone involved in order to maximize purchase. As shown in FIG. 3C, the bore or canal 310 features female threads and is configured to receive the male threads (210, FIG. 2A) of the washer screw (200, FIG. 2A). As shown, the bore portion 310 features a hexagonal portion 311 so that the anchor screw 300 may be driven, as discussed below, into a bone (not shown) via a screw driver or wrench (e.g., Allen wrench) with a corresponding male hexagonal structure.

FIG. 4 is a guide needle 400. As shown, the guide needle 400 is an elongated rod. Suitably, the guide needle 400 is capable of entering the canal or bores of the washer screw 200 and anchor screw 300.

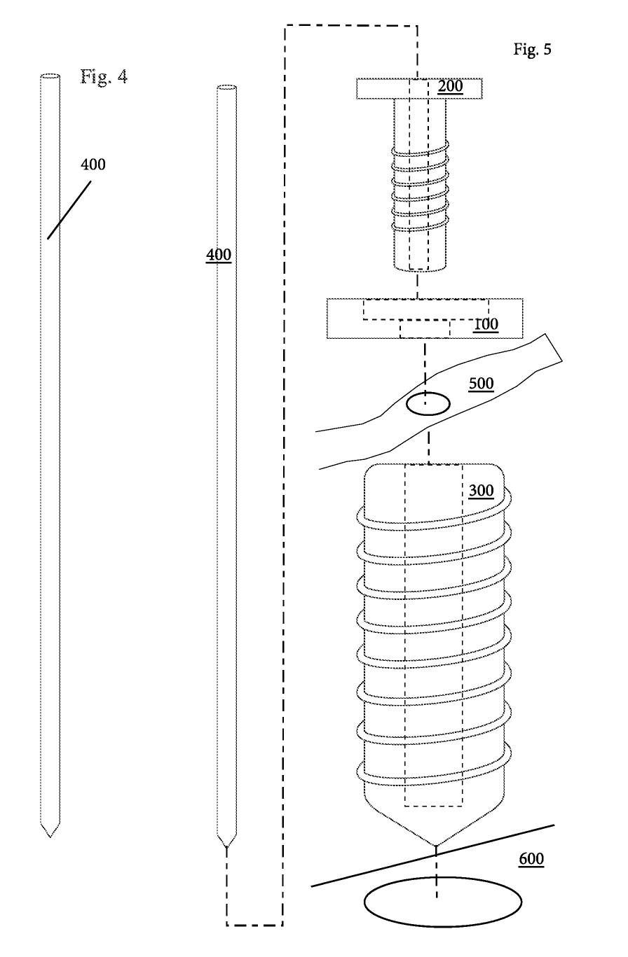

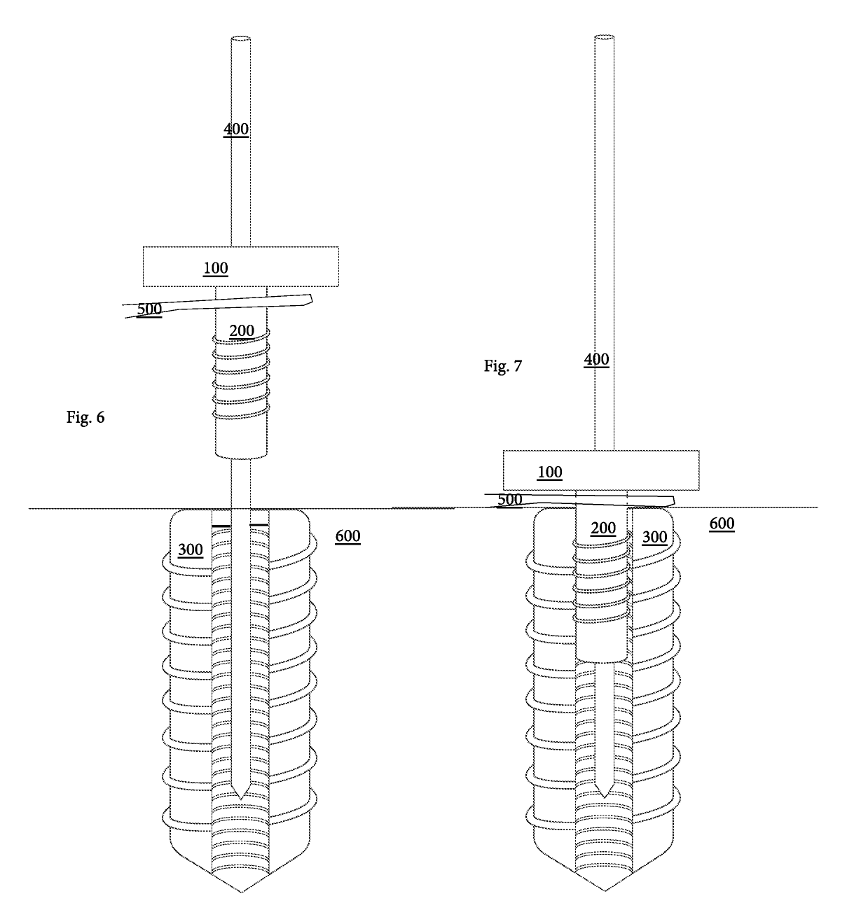

FIGS. 5, 6, and 7 are progressive schematics of the guide needle 400 traversing the anchor screw 300 and the washer screw 200, while the washer screw 200 traverse the soft tissue 500 and the washer 100, and while the anchor screw 300 is deposited into a bone 600. FIGS. 5, 6, and 7 show the guide needle 400 guiding the washer screw 200 into the anchor screw 300. Suitably, the initial step can require insertion of the anchor. screw 300 into the bone 600. After insertion of the anchor screw 300 into the bone 600, the soft tissue 500 may be brought over the anchor screw 300. Third, the guide needle 400 may be used to penetrate the soft tissue 500 before the needle 400 enters the bore portion (310, FIG. 3C) of anchor screw 300. As a fourth step, the washer screw 200, which is cannulated by a bore (230, FIG. 2B), and the washer 100 are inserted onto the guide wire or needle 400 which directs the washer 100 and washer screw 200 into the bore portion (310, FIG. 3C) of anchor screw 300. In a preferred embodiment, the threads 210 of washer screw 200 will engage with internal threads (310, FIG. 3C) of anchor screw as the washer screw 200 is tightened. The interposed soft tissue 500 is suitably captured by the washer 100 and thereby fixed onto the bone 600.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

### PAPER “SEQUENCE LISTING”

Not applicable.

I claim:

1. Interconnecting cannulated screws for attachment of soft tissue to bone.

2. The interconnecting cannulated screws of claim 1 comprising:
an anchor screw with male threads and a threaded female receptacle; and,
a washer screw with male threads, an internal bore through the male threads, and a head.

3. The interconnecting cannulated screws of claim 2 further comprising:
a washer with a central aperture

4. The interconnecting cannulated screws of claim 3 further comprising a guide pin disposed in the female receptacle, through the central aperture of the washer, and through the bore in the washer screw wherein the female receptacle of the anchor screw, the aperture of the washer, and the bore of the washer screw define a cannulated mechanism.

5. The interconnecting cannulated screws of claim 4 wherein the anchor screw directly purchases into a bone while the washer screw is inserted through the washer and penetrated through the soft tissue before ultimately being threaded into the female receptacle of the anchor screw.

6. The interconnecting cannulated screws of claim 5 wherein the washer screw can be guided by the guide pin through the washer, soft tissue, and anchor screw.

7. The interconnecting cannulated screws of claim 2 wherein the soft tissue is interposed between the washer and the anchor screw while the washer screw holds fast the interposition of the soft tissue

8. A method of attaching soft tissue to bone via use of interconnecting cannulated screws.

9. The method of claim 8 wherein the interconnecting cannulated screws comprise:
an anchor screw with male threads and a threaded female receptacle;
a washer screw with male threads, an internal bore through the male threads, and a head; and
a washer with a central aperture.

10. The method of claim 9 further comprising the steps of:
driving the anchor screw into a bone;
passing a guide pin through the internal bore, through the aperture of the washer, through soft tissue, and into the threaded female receptacle; and,
driving the male threads of the screw into the threaded female receptacle.

11. The method of claim 10 wherein the anchor screw directly purchases into the bone while the washer screw is inserted through the washer and penetrated through the soft tissue before ultimately being threaded into the female receptacle of the anchor screw.
